(19) United States
(12) Patent Application Publication
(10) Pub. No.: US 2004/0177851 A1
(43) Pub. Date: Sep. 16, 2004

(54) Occlusion-proof tracheostomy tube

(76) Inventor: Miguel Acosta, Quartz Hill, CA (US)

Correspondence Address: CHRISTIE, PARKER & HALE, LLP, PO BOX 7068, PASADENA, CA, 91109-7068, US

(21) Appl. No.: 10/800,380

(22) Filed: Mar. 12, 2004

Related U.S. Application Data
(60) Provisional application No. 60/454,433, filed on Mar. 13, 2003.

(51) Int. Cl. A61M 16/00

(52) U.S. Cl. 128/207.14; 128/207.15

(57) ABSTRACT

A fitting for retrofitting to a tracheostomy tube is provided that includes an inlet portion for attachment to an outlet structure of the tracheostomy tube and an outlet portion for extending from the outlet structure of the tracheostomy tube. In this embodiment, the outlet portion is connected to the inlet portion and includes at least one side opening. A tracheostomy tube is also provided that includes an inlet structure for insertion into a patient's trachea and an outlet structure for extending from an opening in the patient's neck. In this embodiment, the outlet structure is connected to the inlet structure and includes at least one side opening. A method of modifying a tracheostomy tube is also provided that includes producing at least one side opening in the outlet structure of the tracheostomy tube.

### CROSS-REFERENCE TO RELATED APPLICATION(S)

This application claims priority under 35 U.S.C. § 119 (e) to U.S. Provisional Application Serial No. 60/454,433, filed on Mar. 13, 2003.

### FIELD OF THE INVENTION

The present invention relates generally to a tracheostomy tube having an outlet structure with a plurality of air passageways for reducing the risk of occlusion of the tracheostomy tube and/or to a fitting for retrofitting to a tracheostomy tube, wherein the fitting includes an outlet portion having of plurality of air passageways for reducing the risk of occlusion of the tracheostomy tube.

### BACKGROUND OF INVENTION

A tracheostomy is a surgical operation that creates an opening into the trachea for insertion of a catheter or tube to facilitate breathing. The tube is commonly referred to as a tracheostomy tube. A typical tracheostomy tube includes an inlet structure for insertion into a patient's trachea that is connected to an outlet structure for extending from an opening in the patient's neck. The outlet structure of a typical tracheostomy tube contains only a single opening, i.e. an outlet end opening, such that the only air passageway in the outlet structure is the outlet end opening. As a result, an obstruction or occlusion of the outlet end opening prevents the passage of air through the tracheostomy tube and creates a significant risk of suffocation of the patient.

For example, the outlet end opening may be occluded by the patient's clothing, neck, or chin, or during sleep by the patient's bedsheets or pillows. In addition, for patients of limited mental competence or for patients who are in a coma, the outlet end opening may be occluded by the patient's own hands, arms or fingers. As a result, for these patients twenty-four hour supervision is typically required to ensure that the patient does not suffocate.

Accordingly, a need exists for a tracheostomy tube having an outlet structure that contains a plurality of air passageways to reduce the risk of occlusion of the tracheostomy tube and thereby reduce the risk of suffocation of the patient. Alternatively, or in addition, a need exists for a fitting for retrofitting to a tracheostomy tube, wherein the fitting includes an outlet portion having a plurality of air passageways to reduce the risk of occlusion of the tracheostomy tube and thereby reduce the risk of suffocation of the patient.

### SUMMARY OF THE INVENTION

In one embodiment, the present invention includes a fitting for retrofitting to a tracheostomy tube, wherein the fitting includes an inlet portion for attachment to an outlet structure of the tracheostomy tube and an outlet portion for extending from the outlet structure of the tracheostomy tube. In this embodiment, the outlet portion is connected to the inlet portion and includes at least one side opening.

Another embodiment of the present invention includes a tracheostomy tube having an inlet structure for insertion into a patient's trachea and an outlet structure for extending from an opening in the patient's neck. In this embodiment, the outlet structure is connected to the inlet structure and includes at least one side opening.

Another embodiment of the present invention includes a method of modifying a tracheostomy tube, wherein the tracheostomy tube includes an inlet structure for insertion into a patient's trachea and an outlet structure for extending from an opening in the patient's neck. The method includes producing at least one side opening in the outlet structure of the tracheostomy tube.

### BRIEF DESCRIPTION OF THE DRAWINGS

These and other features and advantages of the present invention will be better understood by reference to the following detailed description when considered in conjunction with the accompanying drawings wherein:

FIG. 1 is a side view of a fitting according to one embodiment of the present invention for retrofitting to a tracheostomy tube;

FIG. 2 is a plan view of the fitting of FIG. 1;

FIG. 3 is a longitudinal cross-sectional view of the fitting of FIG. 1, taken from line 3-3 of FIG. 1;

FIG. 4 is a lateral cross-sectional view of the fitting of FIG. 1, taken from line 4-4 of FIG. 1;

FIG. 5 is a side view of the fitting of FIG. 1 and a tracheostomy tube to which the fitting is attached;

FIG. 6 is a side view of the fitting of FIG. 5 connected to the tracheostomy tube of FIG. 5;

FIG. 6A is a side view of the fitting of FIG. 5 connected to the tracheostomy tube of FIG. 5, showing a filter attached to the fitting;

FIG. 7 is a side view of a tracheostomy tube according to another embodiment of the present invention; and

FIG. 7A is a side view of a tracheostomy tube of FIG. 7, showing a filter attached to an outlet structure of the tracheostomy tube.

### DETAILED DESCRIPTION OF EMBODIMENTS OF THE INVENTION

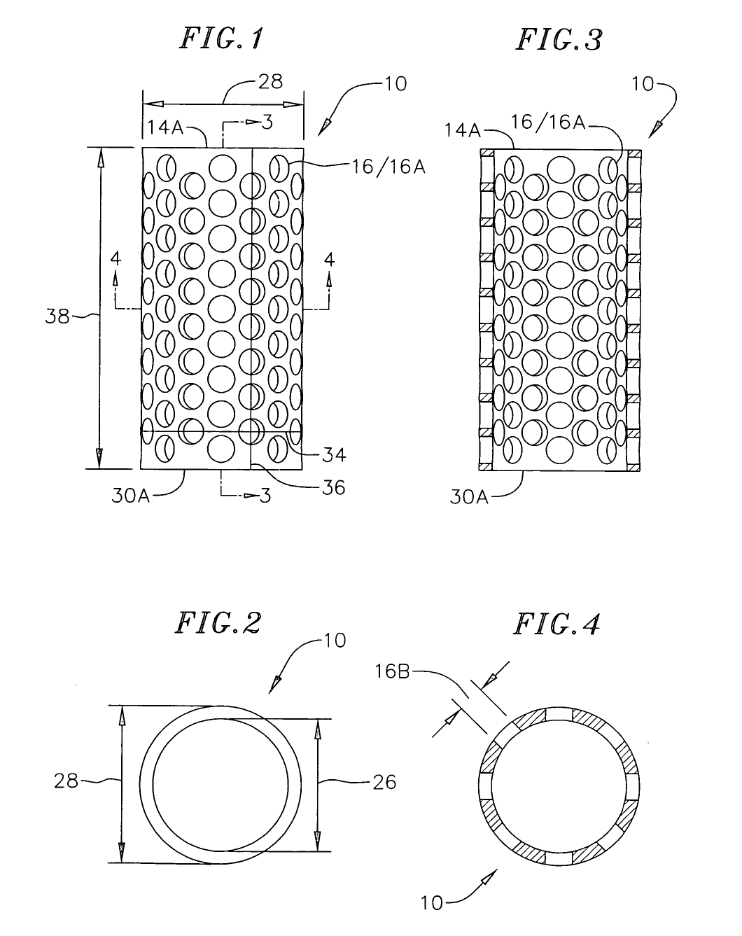

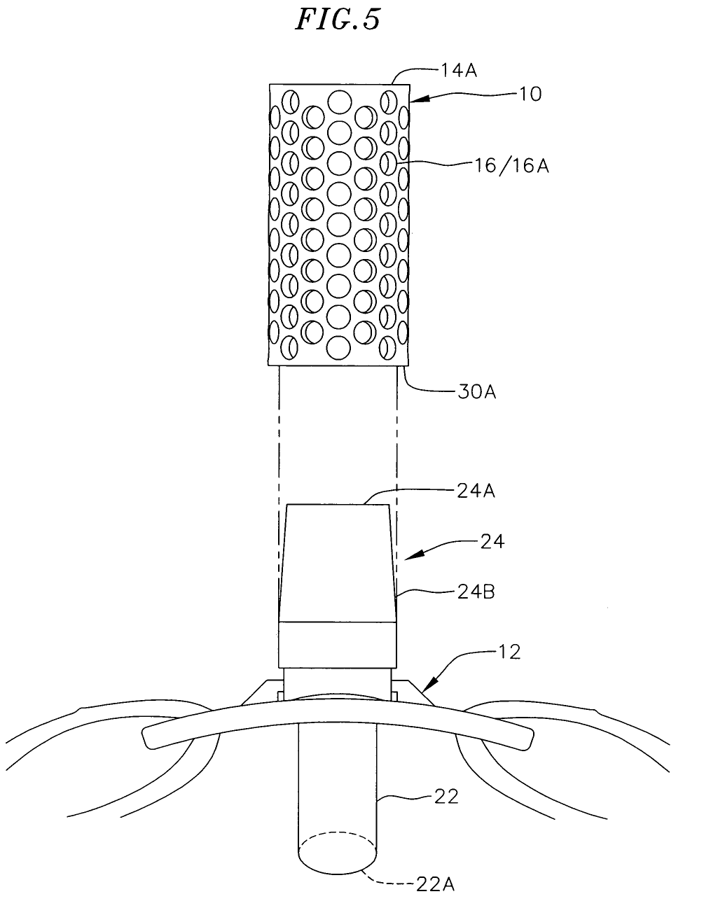

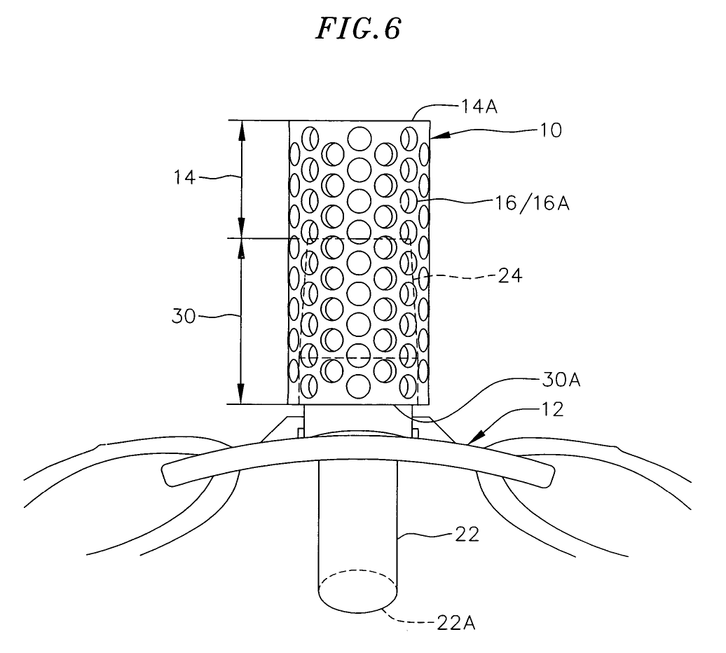

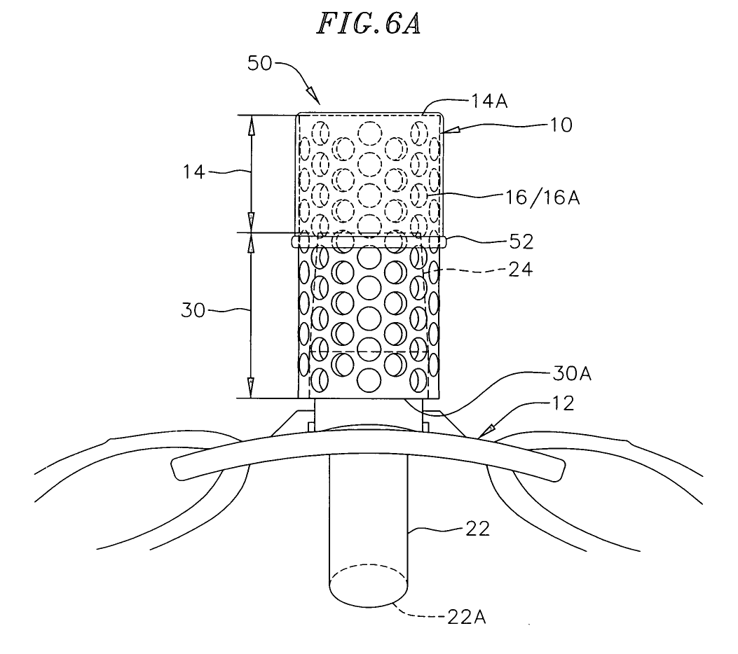

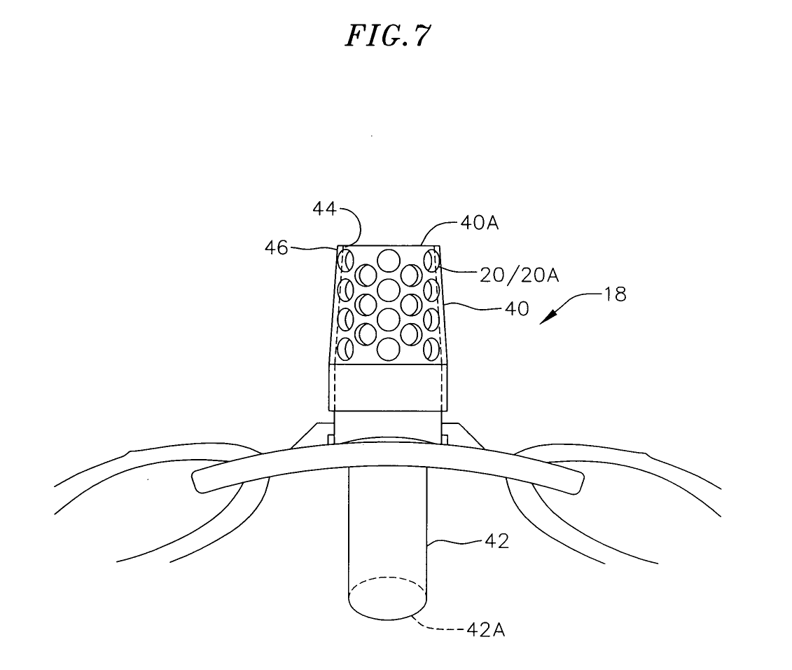

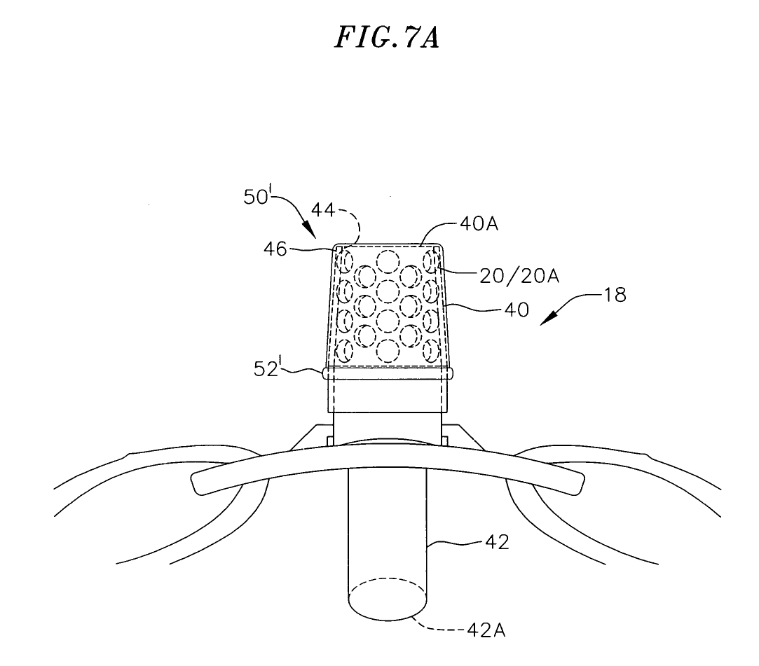

FIGS. 1-6A show a fitting 10 according to one embodiment of the present invention, such as a tube-shaped fitting, for retrofitting to a tracheostomy tube 12 (as shown in FIGS. 5 and 6), wherein the fitting 10 includes an outlet portion 14 having a plurality of air passageways 16A for reducing the risk of occlusion of the tracheostomy tube 12. FIGS. 7 and 7A show a tracheostomy tube 18, according to another embodiment of the present invention, having an outlet structure 40 that contains a plurality of air passageways 20A for reducing the risk of occlusion of the tracheostomy tube 18.

FIGS. 1-6 show a fitting 10 for retrofitting to a tracheostomy tube 12 (shown in FIGS. 5 and 6). In general, the tracheostomy tube 12 may be any tracheostomy tube known in the art, such as that disclosed in U.S. Pat. Nos. 5,067,496 and 4,852,565 issued to Eisele, U.S. Pat. No. 4,315,505 issued to Crandall, and U.S. Pat. No. 4,064,882 issued to Johnson.

The tracheostomy tube 12 of FIGS. 5 and 6 includes an inlet structure 22 for insertion into a patient's trachea that is connected to an outlet structure 24 for extending from an opening in the patient's neck. The inlet structure 22 includes an inlet end opening 22A and the outlet structure 24 includes an outlet end opening 24A.

As shown in FIG. 6, the fitting 10 includes an inlet portion 30 for attachment to the outlet structure 24 of the tracheostomy tube 12 and an outlet portion 14 for extending from the outlet structure 24 of the tracheostomy tube 12. In one embodiment, the inlet portion 30 is removably attached to the outlet structure 24 of the tracheostomy tube 12 by any one of a variety of means, such as a press-fit connection, a snap fit connection, or a frictional connection, among other appropriate attachment means.

In the embodiment of FIGS. 1-6, the fitting 10 is substantially cylindrical and includes an inner diameter 26, and an outer diameter 28 (as shown in FIG. 2 and 4), wherein the inner diameter 26 defines an inlet end opening 30A in the inlet portion 30 of the fitting 10 and an outlet end opening 14A in the outlet portion 14 of the fitting 10. This embodiment may be used when the outlet structure 24 of the tracheostomy tube 12 is substantially cylindrical and includes an outer diameter 24B, as shown in FIG. 5. In one embodiment, the inner diameter 26 of the fitting 10 is smaller than or approximately equal to the outer diameter 24B of the outlet structure 24 of the tracheostomy tube 12, such that the inlet portion 30 of the fitting 10 may be removably secured to the outer diameter 24B of the outlet structure 24 by sliding the inlet portion 30 of the fitting 10 over the outer diameter 24B of the outlet structure 24 to create a frictional connection.

In one exemplary embodiment, the inner diameter 26 is approximately ⅝ of an inch and the outer diameter 28 is approximately ¾ of an inch. However, in alternative embodiments, the inner diameter 26 may have any size appropriate for attachment of the inlet portion 30 to the outlet structure 24 of the tracheostomy tube 12 and the outer diameter 28 may have any size appropriate for providing an adequate amount of stability to the fitting 10. In addition, although the fitting 10 has been described as substantially cylindrical, having the inner diameter 26 and the outer diameter 28, in alternative embodiments the fitting 10 may have any shape suitable for connecting the inlet portion 30 of the fitting 10 to the outlet structure 24 of the tracheostomy tube 12.

The outlet portion 14 of the fitting 10 includes at least one side opening 16 extending between the inner diameter 26, and the outer diameter 28 of the fitting 10. For example, in the embodiment depicted in FIGS. 1-6, the outlet portion 14 of the fitting 10 includes a plurality of side openings 16, wherein each opening creates an air passageway 16A between the inlet end opening 22A of the inlet structure 22 of the tracheostomy tube 12 and the outlet portion 14 of the fitting 10.

Although, FIG. 6 shows openings 16 in the inlet portion 30 and in the outlet portion 14 of the fitting 10, it is not necessary that the inlet portion 30 contains openings 16. However, the inlet portion 30 may include openings 16 to account for variations in the length of the outlet structure of the various tracheostomy tubes to which the fitting 10 may be attached, and to allow for the length of the fitting 10 that extends from the outlet structure of the tracheostomy tube, to which the fitting 10 is attached, to vary. In one embodiment, the overall length 38 of the fitting 10 is approximately 1 and ¼ inches. Although, in alternative embodiments, the overall length 38 of the fitting 10 may be any length appropriate for allowing an adequate amount of the fitting 10 to extend from the outlet structure 24 of the tracheostomy tube 12.

In the embodiments of FIGS. 1-6, the plurality of openings 16 are arranged in a plurality of rows 34 and a plurality of columns 36. In the depicted embodiment, the openings 16 are substantially circular, having a diameter 16B of approximately ⅛ of an inch. However, in alternative embodiments, the openings 16 may be any shape or size as long as the openings 16 create appropriate air passageways 16A. Although the plurality of rows 34 and a plurality of columns 36 may be approximately horizontally and vertically aligned, respectively, in the depicted embodiment, each row 34 is horizontally offset from each adjacent row and each column 36 is vertically offset from each adjacent column. The offsetting of the rows 34 and columns 36 facilitates reducing the risk of occlusion of the tracheostomy tube 12. In one embodiment the fitting 10 is composed of a strong flexible material plastic.

FIG. 7 shows a tracheostomy tube 18 according to another embodiment of the present invention. The tracheostomy tube 18 may be any tracheostomy tube known in the art, such as those disclosed in the above listed U.S. Patents, as long as the tracheostomy tube 18 includes an outlet structure 40 as described in detail below.

The tracheostomy tube 18 of FIG. 7 includes an inlet structure 42, having an inlet end opening 42A, for insertion into a patient's trachea that is connected to an outlet structure 40 for extending from an opening in the patient's neck. In one embodiment, the outlet structure 40 is substantially cylindrical and includes an inner diameter 44, an outer diameter 46 and at least one side opening 20 extending from the inner diameter 44 to the outer diameter 46.

For example, in the depicted embodiment the outlet structure 40 includes a plurality of side openings 20, wherein each opening 20 creates an air passageway 20A between the inlet end opening 42A of the inlet structure 42 and the outlet structure 40. The openings 20 may contain each of the shapes, sizes and arrangements as described above for the openings 16 in the fitting 10. In addition, the outlet structure 40 may also include an outlet end opening 40A defined by the inner diameter 44 of the outlet structure 40, which creates another passageway between the inlet end opening 42A of the inlet structure 42 and the outlet structure 40.

Although the outlet structure 40 of the tracheostomy tube 18 has been described as substantially cylindrical, having the inner diameter 44 and the outer diameter 46, in alternative embodiments the outlet structure 40 may have any shape and size suitable for the passage of air between the inlet end opening 42A of the inlet structure 42 and the outlet structure 40.

When a patient breaths through a tracheostomy tube, the air entering the patient's body does not receive the filtering that occurs when a person breaths through the nose. Therefore, when a patient breaths through a tracheostomy tube, the patient is more susceptible to infection by airborne pathogens. As such, in one embodiment, such as that shown in FIG. 6A, a filter 50 is attached to the fitting 10 of the tracheostomy tube 12.

The filter 52 may be composed of any appropriate breathable fabric, such as the material of a typical surgeon's mask. The filter 50 may cover the entire length of the fitting 10. However, it is only necessary that the filter 50 cover the outlet portion 14 of the fitting 10. The filter 50 may be attached to the fitting 10 by any appropriate means. For example, in the depicted embodiment, the filter 50 includes an elastic band 52 that secures the filter to the fitting 10. FIG. 7A shows a filter 50′ having an elastic band 52′ attached to the outlet structure 40 of the tracheostomy tube 18. The filter 50′ shown in FIG. 7A contains the same characteristics as the filter 50 shown in FIG. 6A and described above.

The preceding description has been presented with reference to presently preferred embodiments of the invention. Persons skilled in the art and technology to which this invention pertains will appreciate that alterations and changes in the described structures and methods of operation can be practiced without meaningfully departing from the principle, spirit and scope of this invention. Accordingly, the foregoing description should not be read as pertaining only to the precise structures described and shown in the accompanying drawings, but rather should be read as consistent with and as support for the following claims, which are to have their fullest and fairest scope.

What is claimed is:

1. A fitting for retrofitting to a tracheostomy tube comprising:
an inlet portion for attachment to an outlet structure of the tracheostomy tube; and
an outlet portion for extending from the outlet structure of the tracheostomy tube, wherein the outlet portion is connected to the inlet portion, and wherein the outlet portion comprises at least one side opening.

2. The tracheostomy tube fitting of claim 1, wherein the outlet portion comprises a plurality of rows of side openings and a plurality of columns of side openings.

3. The tracheostomy tube fitting of claim 2, wherein each row is offset from an adjacent row and wherein each column is offset from an adjacent column.

4. The tracheostomy tube fitting of claim 1, further comprising a filter attached to the outlet portion for filtering air that enters the fitting through the at least one side opening.

5. A fitting for retrofitting to a tracheostomy tube comprising:
a hollow cylinder having an inner diameter, an outer diameter, an inlet end opening and an outlet end opening, the cylinder further comprising:
an inlet portion for attachment to an outlet structure of the tracheostomy tube, wherein the inlet portion comprises the inlet end opening; and
an outlet portion for extending from the outlet structure of the tracheostomy tube, wherein the outlet portion is connected to the inlet portion, and wherein the outlet portion comprises the outlet end opening and at least one side opening that extends from the inner diameter to the outer diameter.

6. The tracheostomy tube fitting of claim 5, wherein the outlet portion comprises a plurality of rows of side openings that extend from the inner diameter to the outer diameter and a plurality of columns of side openings that extend from the inner diameter to the outer diameter.

7. The tracheostomy tube fitting of claim 6, wherein each row is offset from an adjacent row and wherein each column is offset from an adjacent column.

8. The tracheostomy tube fitting of claim 5, further comprising a filter attached to the outlet portion for filtering air that enters the fitting through the at least one side opening.

9. A tracheostomy tube comprising:
an inlet structure for insertion into a patient's trachea; and
an outlet structure for extending from an opening in the patient's neck, wherein the outlet structure is connected to the inlet structure, and wherein the outlet structure comprises at least one side opening.

10. The tracheostomy tube of claim 9, wherein the outlet structure comprises a plurality of rows of side openings and a plurality of columns of side openings.

11. The tracheostomy tube of claim 10, wherein each row is offset from an adjacent row and wherein each column is offset from an adjacent column.

12. The tracheostomy tube of claim 9, further comprising a filter attached to the outlet structure for filtering air that enters the tracheostomy tube through the at least one side opening.

13. A tracheostomy tube comprising:
an inlet structure for insertion into a patient's trachea; and
an outlet structure for extending from an opening in the patient's neck, wherein the outlet structure is connected to the inlet structure, and wherein the outlet structure comprises an inner diameter, an outer diameter and at least one side opening that extends from the inner diameter to the outer diameter.

14. The tracheostomy tube of claim 13, wherein the outlet structure comprises a plurality of rows of side openings that extend from the inner diameter to the outer diameter and a plurality of columns of side openings that extend from the inner diameter to the outer diameter.

15. The tracheostomy tube of claim 14, wherein each row is offset from an adjacent row and wherein each column is offset from an adjacent column.

16. The tracheostomy tube of claim 13, further comprising a filter attached to the outlet structure for filtering air that enters the tracheostomy tube through the at least one side opening.

17. A method of modifying a tracheostomy tube that comprises an inlet structure for insertion into a patient's trachea and an outlet structure for extending from an opening in the patient's neck, the method comprising:
producing at least one side opening in the outlet structure of the tracheostomy tube.
